(19) United States
(12) Patent Application Publication
(10) Pub. No.: US 2004/0097936 A1
(43) Pub. Date: May 20, 2004

(54) Set of osteosynthesis plates -5 variants of the same basic type

(76) Inventor: Rainer Ebid, Taufkirchen (DE)

Correspondence Address: Rainer Ebid, Eichenstr. 3, Taufkirchen, 82024, DE

(21) Appl. No.: 10/294,832

(22) Filed: Nov. 15, 2002

(51) Int. Cl. A61B 17/58

(52) U.S. Cl. 606/69

(57) ABSTRACT

A set of osteosynthesis plates with 5 different variants of the same basic type (triangular shape) of plates is introduced. The size of any of the 5 variants may vary. The thickness of any of the plates may be equal all over the plate or vary in the different parts of the same plate.

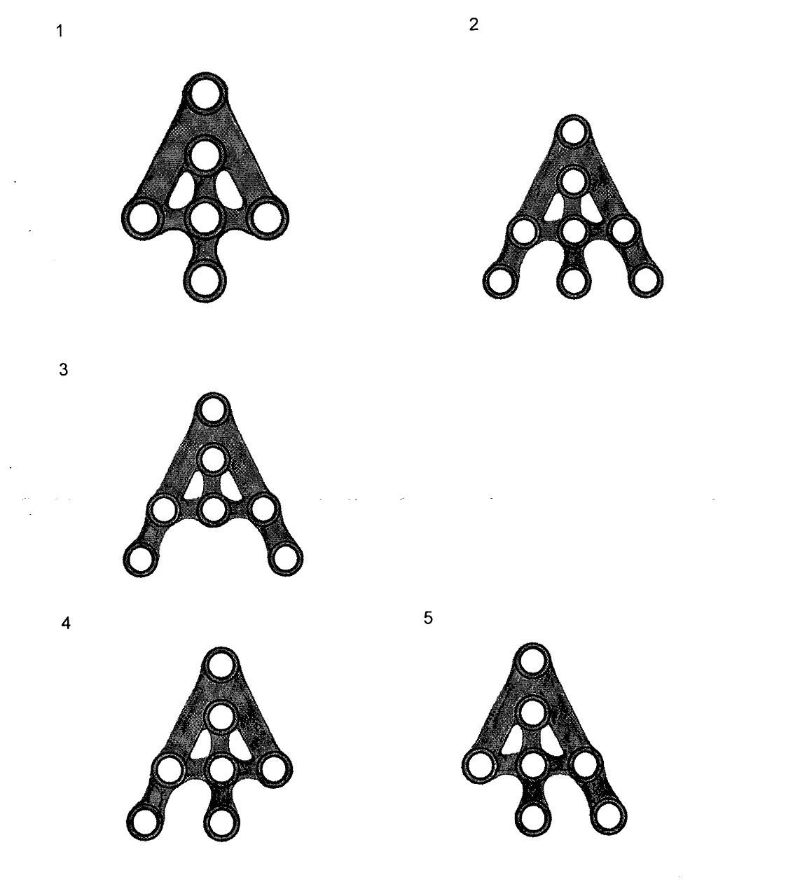

The osteosynthesis plates are designed to be applied on the medical and veterinary field.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING

Not Applicable

### BACKGROUND OF THE INVENTION

A new type of osteosynthesis plates comprising 5 variants of the same basic type (triangular shape) of plates is introduced as a set. The plates serve to immobilise bones or parts of bones in relation to each other with the help of screws.

The plates are designed to be applied on the medical and veterinary field.

The experience shows, that certain fractures use to dislocate in spite of hitherto known immobilisation techniques.

### BRIEF SUMMARY OF THE INVENTION

This type of plate, 5 variants of the same basic principle, shall especially withstand rotary forces. Therefore the triangular shape was constructed. The arrangement of the screw-holes allows to use this type of plate in an area with little space for the fixation of the plate. The plate is not bound to any specific type of osteosynthesis, but may be applied wherever suitable in human and veterinary medicine.

As to be seen in the drawings any of the plates has round screw-holes. Any of the screw-holes may be oval as well, or have any kind of angular shape.

The Basic shape of the plate is an isosceles triangle with one screw-hole on the top (A) and one screw-hole at each end of the legs of equal length (B (left) and C (right)). The third leg contains the screw-hole E, with equal distance to B and C. A and C are linked together by a “middle-leg”, which creates 2 equivalent triangles. In the middle between A and E, the screw-hole D is to be found. The distances between A and D, D and E, B and E, and C and E are all equal. This is the common basic shape for all of the 5 variants within the set of osteosynthesis plates.

To create the variants, the following extensions are made: The legs of equal length of the triangle are elongated, as well as the “middle-leg”. At the end of each leg a screw-hole exists. G on the left side, H on the right side, F on the middle leg. All of those screw-holes are on one line, which is parallel to the leg with the screw-holes B, C and E. The distance between E and F is as long as between A and D.

In 1G and H are lacking, 2 is the variant just described above, in 3F is lacking, in 4H is lacking, and in 5G is lacking. In this order the drawings are arranged.

Any of the corners of one and the same plate may be round or angular. Any of the legs and/or the “middle-leg” may be broadened in the area of the screw-holes to maintain enough material around any of the screw-holes. On the drawings plates with broadened areas in the areas of the screw-holes are seen.

The drawings show a direct view on the plates with their round screw-holes. The size of any of the variants of the plates may be variable. The thickness of any of the variants of the plates may be equal in any of its parts or may vary in the different parts of the same plate.

Even if the plates are not bound to any specific area for usage in the human body or in the body of an animal, there is one special site, where it may serve due to hitherto experienced problems. In collum mandibulae fractures (the collum mandibulae is a part of the lower jaw) an application seems to be advantageous.

### LEGENDS OF THE DRAWINGS

The drawings show the direct view on 5 single osteosynthesis plates (5 variants of the same basic type) including their round screw-holes.

The size of any of the plates may vary.

The thickness of any of the plates may be equal in any of its parts or may vary in the different parts of one plate.

What I claim as my invention is:

1. the new form of osteosynthesis plates (triangular shape) including all of the 5 introduced variants, as described.

2. My claim extends to different sizes as well as to the thickness of any of the plates.
This thickness may be equal all over the same plate or vary in the different parts of the same plate.

3. My claim extends also to any conceivable material the plates may be produced of.
